(19) United States
(12) Patent Application Publication
(10) Pub. No.: US 2019/0033435 A1
(43) Pub. Date: Jan. 31, 2019

(54) ULTRASOUND DIAGNOSTIC DEVICE, IMAGE PARAMETER SETTING METHOD AND STORAGE MEDIUM

(71) Applicant: Konica Minolta, Inc., Chiyoda-ku Tokyo (JP)

(72) Inventor: Tomohito SAKAI, Yokohama-shi Kanagawa (JP)

(21) Appl. No.: 16/050,478

(22) Filed: Jul. 31, 2018

(30) Foreign Application Priority Data
Jul. 31, 2017 (JP) 2017-147649
Jan. 17, 2018 (JP) 2018-005313

(51) Int. Cl. G01S 7/52; A61B 8/08; G01S 15/89

(52) U.S. Cl. CPC G01S 7/52085; G01S 7/52025; G01S 15/8952; G01S 7/52057; A61B 8/5207

(57) ABSTRACT

An ultrasound diagnostic device, including: a transmitter which outputs a drive signal to an ultrasound probe that transmits and receives ultrasound waves; a receiver which obtains a reception signal from the ultrasound probe; a first hardware processor which generates ultrasound image data from the reception signal and performs image processing to the generated ultrasound image data; an operator which receives input of a depth of an ultrasound image to be displayed; and a second hardware processor which obtains a plurality of preset image parameters corresponding to the input depth, and controls the transmitter, the receiver and the first hardware processor to generate the ultrasound image data corresponding to the input depth according to the obtained plurality of image parameters.

### BACKGROUND

### Cross-Reference to Related Application

The present application claims priority under 35 U.S.C. § 119 to Japanese Patent Application No. 2017-147649, filed on Jul. 31, 2017 and Japanese Patent Application No. 2018-005313, filed on Jan. 17, 2018, the entire contents of which are incorporated herein by reference.

### 1. Technological Field

The present invention relates to an ultrasound diagnostic device, an image parameter setting method and a storage medium.

### 2. Description of the Related Art

In ultrasound diagnosis, examination can be performed repeatedly since the heart beat or the movement of a fetus is obtained as an ultrasound image by an easy operation of putting the ultrasound probe against the body surface, and furthermore the patient is safe. There is known an ultrasound diagnostic device which is used for the ultrasound diagnosis and generates and displays the ultrasound image.

The ultrasound diagnostic device has many image parameters as setting information for generating ultrasound image data. It is possible to obtain good ultrasound image data by setting image parameters according to the depth, the site and the subject.

For example, there is known an ultrasound diagnostic device which changes the drive frequency of ultrasound waves to be transmitted according to the visual field depth and obtains a diagnostic image having an excellent resolution or a diagnostic image which is easy to see (see Japanese Patent No. 3947647).

There is also known an ultrasound diagnostic imaging system which forms a spatially-combined image by combining multiple different visual directions, the number of the different visual directions being changeable according to the change in image depth as an operation parameter (see Japanese Patent No. 4694692).

However, in the above conventional ultrasound diagnostic device and the ultrasound diagnostic imaging system, there has been a problem that, in a case where the depth is changed, changeable parameters are limited, and the brightness balance, contrast and followability are unintentionally changed or deteriorated in some cases due to a partial change in the image parameters and inconsistency with the image parameter which is not changed before and after the depth change. In order to correct them, the operating person needs to change the image parameters, which is a troublesome operation.

### SUMMARY

An object of the present invention is to easily obtain good ultrasound image data according to a plurality of image parameters without the troublesome operation.

To achieve at least one of the abovementioned objects, according to a first aspect of the present invention, an ultrasound diagnostic device reflecting one aspect of the present invention includes: a transmitter which outputs a drive signal to an ultrasound probe that transmits and receives ultrasound waves; a receiver which obtains a reception signal from the ultrasound probe; a first hardware processor which generates ultrasound image data from the reception signal and performs image processing to the generated ultrasound image data; an operator which receives input of a depth of an ultrasound image to be displayed; and a second hardware processor which obtains a plurality of preset image parameters corresponding to the input depth, and controls the transmitter, the receiver and the first hardware processor to generate the ultrasound image data corresponding to the input depth according to the obtained plurality of image parameters.

According to a second aspect of the present invention, an image parameter setting method in an ultrasound diagnostic device including: a transmitter which outputs a drive signal to an ultrasound probe that transmits and receives ultrasound waves; a receiver which obtains a reception signal from the ultrasound probe; and a hardware processor which generates ultrasound image data from the reception signal and performs image processing to the generated ultrasound image data, the image parameter setting method reflecting one aspect of the present invention includes: a step of receiving input of a depth of an ultrasound image to be displayed; and a step of obtaining a plurality of preset image parameters corresponding to the input depth, and controlling the transmitter, the receiver and the hardware processor to generate the ultrasound image data corresponding to the input depth according to the obtained plurality of image parameters.

According to a third aspect of the present invention, a computer readable storage medium reflecting one aspect of the present invention stores a program for causing a computer to function as: a transmitter which outputs a drive signal to an ultrasound probe that transmits and receives ultrasound waves; a receiver which obtains a reception signal from the ultrasound probe; a first hardware processor which generates ultrasound image data from the reception signal and performs image processing to the generated ultrasound image data; an operator which receives input of a depth of an ultrasound image to be displayed; and a second hardware processor which obtains a plurality of preset image parameters corresponding to the input depth, and controls the transmitter, the receiver and the first hardware processor to generate the ultrasound image data corresponding to the input depth according to the obtained plurality of image parameters.

### BRIEF DESCRIPTION OF THE DRAWINGS

The advantages and features provided by one or more embodiments of the invention will become more fully understood from the detailed description given hereinafter and the appended drawings which are given by way of illustration only, and thus are not intended as a definition of the limits of the present invention, and wherein:

FIG. 1 is an outer appearance view of an ultrasound diagnostic device in an embodiment of the present invention;

FIG. 2 is a block diagram showing the functional configuration of the ultrasound diagnostic device;

FIG. 3 is a view showing an example of an image parameter set group;

FIG. 4 is a flowchart showing image parameter set creation processing;

FIG. 5 is a view showing an image parameter set creation screen;

FIG. 6 is a view showing an image parameter copy window;

FIG. 7 is a view showing an image parameter set of all shallow copy and all deep copy;

FIG. 8 is a view showing an image parameter set of one-step shallow copy and one-step deep copy;

FIG. 9 is a view showing a storage dialog;

FIG. 10 is a flowchart showing image parameter set using processing;

FIG. 11 is a flowchart showing image parameter set change processing;

FIG. 12A is a view showing a B-mode image of the depth 2 [cm] of a first image parameter set;

FIG. 12B is a view showing a B-mode image of the depth 4 [cm] of a first image parameter set;

FIG. 12C is a view showing a B-mode image of the depth 7 [cm] of a first image parameter set;

FIG. 13A is a view showing a B-mode image of the depth 3 [cm] of a first image parameter set;

FIG. 13B is a view showing a B-mode image of the depth 3 [cm] for which the observation site of the first image parameter set is changed; and

FIG. 13C is a view showing a B-mode image of the depth 3 [cm] of a second image parameter set.

### DETAILED DESCRIPTION OF THE EMBODIMENTS

Hereinafter, one or more embodiments of the present invention will be described in detail with reference to the drawings. However, the scope of the invention is not limited to the disclosed embodiments.

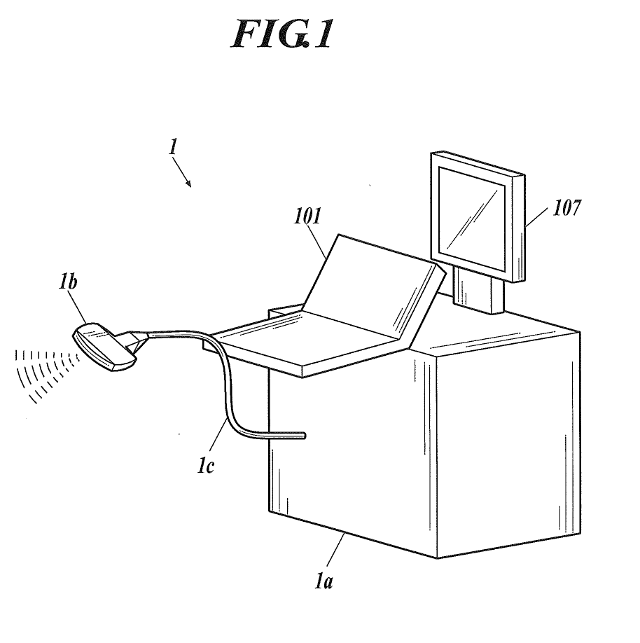

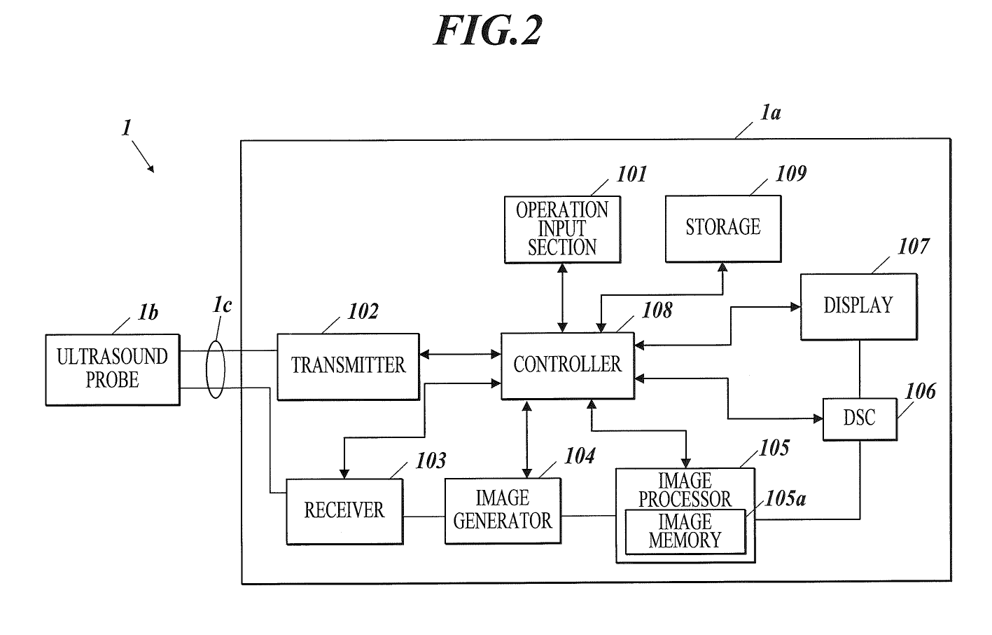

The embodiment will be described with reference to FIG. 1 to FIG. 13C. First, the device configuration in the embodiment will be described with reference to FIG. 1 and FIG. 2. FIG. 1 is an outer appearance view of an ultrasound diagnostic device 1 in the embodiment. FIG. 2 is a block diagram showing the functional configuration of the ultrasound diagnostic device 1.

As shown in FIG. 1 and FIG. 2, the ultrasound diagnostic device 1 in the embodiment includes an ultrasound diagnostic device main body 1a and an ultrasound probe 1b. The ultrasound probe 1b transmits ultrasound waves (transmission ultrasound waves) to a subject such as a living body not shown in the drawings, and receives reflected waves of the ultrasound waves (reflected ultrasound waves) which were reflected in the subject. The ultrasound diagnostic device main body 1a is connected to the ultrasound probe 1b via a cable 1c, causes the ultrasound probe 1b to transmit the transmission ultrasound waves to the subject by transmitting drive signals of electrical signals to the ultrasound probe 1b, and performs imaging of the internal state of the subject as an ultrasound image on the basis of the reception signals which are electrical signals generated in the ultrasound probe 1b according to the reflected ultrasound waves from inside the subject received by the ultrasound probe 1b.

The ultrasound probe 1b includes a plurality of oscillators formed of piezoelectric elements, the oscillators being arranged in one-dimensional array in azimuth direction. In the embodiment, for example, there is used the ultrasound probe 1b including 192 oscillators. The oscillators may be arranged in two-dimensional array. The number of the oscillators may be arbitrarily set. Though the embodiment adopts an electronic scanning probe of the linear scanning type for the ultrasound probe 1b, either of the electronic scanning type or the machine scanning type may be adopted. Any type of the linear scanning type, sector scanning type and convex scanning type may be adopted, and the ultrasound probe 1b may be exchanged with a different type of ultrasound probe 1b.

As shown in FIG. 2, the ultrasound diagnostic device main body 1a includes, for example, an operation input section 101 as an operator, a transmitter 102, a receiver 103, an image generator 104, an image processor 105, a DSC (Digital Scan Converter) 106, a display 107, a controller 108 and a storage 109.

The operation input section 101 includes, for example, various switches, buttons, a dial, a track ball, a mouse, a keyboard and the like for performing a command by an operating person (doctor, technician or the like) instructing start of diagnosis and for inputting data such as personal information of the subject. The operation input section 101 outputs the operation signal to the controller 108. The operation input section 101 further includes a touch panel provided on a display screen of the display 107.

The transmitter 102 is a circuit which causes the ultrasound probe 1b to generate transmission ultrasound waves by supplying drive signals which are electrical signals via the cable 1c to the ultrasound probe 1b in accordance with control by the controller 108. The transmitter 102 includes, for example, a clock generation circuit, a delay circuit and a pulse generation circuit. The clock generation circuit is a circuit for generating clock signals for determining the transmission timing of the drive signals and transmission frequency. The delay circuit is a circuit for setting the delay time for each individual path corresponding to the oscillator for the transmission timing of the drive signals and delaying the transmission of the drive signals by the set delay time to focus the transmission beams formed of the transmission ultrasound waves. The pulse generation circuit is a circuit for generating pulse signals as drive signals at predetermined periods. The transmitter 102 formed as descried above generates, for example, transmission ultrasound waves by driving a part of (for example 64) consecutive oscillators among a plurality of (for example, 192) oscillators arranged in the ultrasound probe 1b. The transmitter 102 performs scanning by shifting, in the azimuth direction, the oscillators which drive at each generation of the transmission ultrasound waves.

The receiver 103 is a circuit which receives reception signals which are electrical signals via the cable 1c from the ultrasound probe 1b in accordance with the control by the controller 108. The receiver 103 includes, for example, an amplifier, an A/D converting circuit and a phasing addition circuit. The amplifier is a circuit for amplifying the reception signals at a preset amplifying rate for each individual path corresponding to the oscillator. The A/D converting circuit is a circuit for performing A/D conversion of the amplified reception signals. The phasing addition circuit is a circuit for aligning the time phases by providing the delay times for the reception signals subjected to the A/D conversion for the respective individual paths corresponding to the respective oscillators, and adding the aligned time phases (phasing addition) to generate sound ray data.

The image generator 104 generates B-mode image data by performing envelop detection processing, log compression and the like to the sound ray data from the receiver 103 and adjusting the dynamic range and the gain to perform brightness conversion, in accordance with control by the controller 108. That is, the B-mode image data represents the strength of the reception signal by brightness. The image generator 104 can generate ultrasound image data of an image mode other than the B-mode. The image generator 104 can generate, for example, ultrasound image data by at least one of a B-mode, an A-mode, an M-mode, a color Doppler mode, a power Doppler mode, a CW (Continuous Wave) mode and an Elastography mode.

The image processor 105 includes an image memory 105a formed of a semiconductor memory such as a DRAM (Dynamic Random Access Memory). In accordance with control by the controller 108, the image processor 105 can perform various types of image processing to the B-mode image data output from the image generator 104, and stores B-mode image data which is subjected to or not subjected to the image processing in the image memory 105a by the unit of frame. Especially, the image processor 105 performs image processing of providing a gain value by after-mentioned TGC (Time Gain Compensation) of a normal operation, offset TGC, gain of a normal operation and offset gain to B-mode image data output from the image generator 104. The TGC is a function of correcting and adjusting the amplification degree (gain) of the received ultrasound waves with respect to the time corresponding to the distance at a certain interval. The image data by the unit of frame may be referred to as ultrasound image data or frame image data. The image processor 105 transmits frame image data stored in the image memory 105a to the DSC 106 in accordance with control by the controller 108.

In accordance with control by the controller 108, the DSC 106 converts the frame image data received from the image processor 105 into an image signal for the display 107 and outputs the converted signal to the display 107.

As the display 107, there can be applied a display device such as an LCD (Liquid Crystal Display), a CRT (Cathode-Ray Tube) display, an organic EL (Electronic Luminescence) display, an inorganic EL display and plasma display. The display 107 performs display of an image on the display screen in accordance with the image signal output from the DSC 106 in accordance with control by the controller 108.

The controller 108 includes, for example, a CPU (Central Processing Unit), a ROM (Read Only Memory) and a RAM (Random Access Memory), reads out various processing programs such as a system program or the like stored in the ROM and loads them onto the RAM, and integrally controls the operations of the sections of the ultrasound diagnostic device 1 in accordance with the loaded programs. The ROM is formed of a nonvolatile memory or the like such as a semiconductor, and stores a system program corresponding to the ultrasound diagnostic device 1, various processing programs executable on the system program, various types of data and the like such as a gamma table. Especially, the various programs include, for example, an image parameter set creation program for executing image parameter set creation processing, an image parameter set using program for executing the image parameter set using processing, an image parameter set changing program for executing the image parameter set change processing and the like. These programs are stored in a form of computer readable program code, and the CPU sequentially executes the operation according to the program code. The RAM forms a working area for temporarily storing various programs executed by the CPU and data according to these programs.

The storage 109 is formed of a large-capacity storage medium such as an HDD (Hard Disk Drive), for example, and stores ultrasound image data, an after-mentioned image parameter set group 200, various types of setting information and the like so as to be linked with patient information.

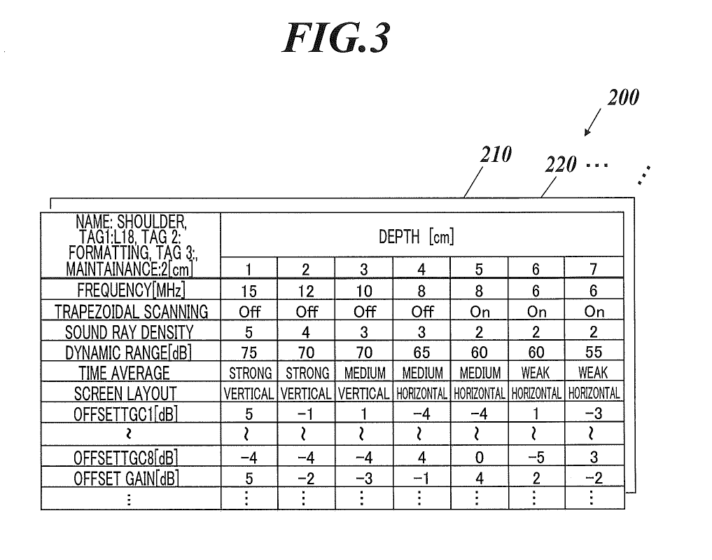

Next, with reference to FIG. 3, the configuration of the image parameter set group 200 stored in the storage 109 will be described. FIG. 3 is a view showing an example of the image parameter set group 200.

The image parameter set group 200 has a plurality of image parameter sets as tables for storing setting values of a plurality of image parameters corresponding to a plurality of depths. As shown in FIG. 3, the image parameter set group 200 has image parameter sets 210, 220 . . . which are provided by the observation site and the use such as ultrasound diagnosis.

The image parameter set 210 has a name, tag 1, tag 2, tag 3 and maintenance information of the image parameter set. The name, tag 1, tag 2 and tag 3 function as identification information of the image parameter set 210. Thus, for example, a same name may be provided to a plurality of image parameter sets, and a different tag may be provided to each image parameter set. Though up to three tags can be registered here, the number of tags is not limited to this. The image parameter set may have an identification number.

The name is a name of image parameter set, and may be an observation site, for example. The tags may be freely set by an operating person to be the type of the ultrasound probe 1b in the ultrasound diagnosis using the image parameter set, the field of the ultrasound diagnosis and the like. The name and tags are also used for keyword search and tag search of the image parameter set.

The maintenance information is information on whether to maintain the last depth when the image parameter set to be changed is selected in a state in which the image parameter set is not selected or another image parameter set is selected, and information on the depth after change in a case where the depth is not maintained (the depth is changed). The maintenance information is, for example, “On (in a case of maintaining the depth)” and “x ([cm], x: arbitrary depth value (in a case of not maintaining the depth))”.

The image parameter set 210 contains setting values of (transmission) frequency, trapezoidal scanning, sound ray density, dynamic range, time average, screen layout, offset TGC 1 to 8, offset gain and the like as image parameters of the B-mode corresponding to a plurality of depths (for example, 1 to 7 [cm] (1 [cm] interval)). Though not shown in the drawings, the image parameter set 210 has setting values of image parameters corresponding to the plurality of depths (for example, 1 to 7 [cm]) also for other modes such as an M-mode.

The (transmission) frequency is the frequency [MHz] of the transmission ultrasound waves. The trapezoidal scanning is information indicating whether to perform trapezoidal scanning of generating trapezoidal B-mode image data by changing the angle of the sound ray of each oscillator using the linear ultrasound probe 1b. The sound ray density is information indicating the density of sound ray of the transmission ultrasound waves emitted from the ultrasound probe 1b.

The dynamic range is information indicating the dB amount to be assigned to the brightness tone of generated image data among the brightness of sound ray data exceeding 100 dB. The time average is the amount indicating the number of temporal consecutive frames of B-mode image (each pixel value) to have arithmetic average or weighting average in the live B-mode image. The time average in the embodiment is represented as strong, medium and weak. The screen layout is information indicating whether to perform vertical display or horizontal display in a case of double-screen display.

The offset TGC 1 to 8 is information indicating the gain value (brightness value) [dB] to be added to each area as offset which is the correction value to the time corresponding to the distance of each area that is obtained by dividing the B-mode image into eight areas in the depth direction. For example, in the embodiment, the TGC by a normal operation and the offset TGC is a same area. The number of areas of offset TGC and normal TGC is not limited to eight. In a case where the TGC of normal operation is done to increase or decrease the brightness value of each area of the displayed B-mode image by input of the operating person, if the offset TGC is valid, the brightness value of normal TGC+offset TGC is added to each pixel value of each corresponding area of the B-mode image.

The offset gain is information indicating the gain value (brightness value) [dB] to be added as offset to the entire area of the B-mode image. In a case where gain adjustment of normal operation is done to increase or decrease the brightness value of each area of the displayed B-mode image by input of normal gain by the operating person, if the offset gain is valid, normal gain adjustment value+offset gain brightness value are added to each pixel value of the entire area of the B-mode image.

In the image parameter set 210, the setting value of trapezoidal scanning of image parameter is invalid (off) when the depth is shallow, and the setting value is valid (on) when the depth is deep. The setting value of trapezoidal scanning of image parameter may be configured so that the angle of sound ray is small when the depth is shallow and the angle of sound ray is large when the depth is deep.

In the image parameter set 210, the setting value of sound ray density of image parameter is high when the depth is shallow and the setting value is low when the depth is deep. In the image parameter set 210, the setting value of time average of image parameter is strong when the depth is shallow and the setting value is weak when the depth is deep.

In the image parameter set 210, the setting value of dynamic range of image parameter is high when the depth is shallow and the setting value is low when the depth is deep. In the image parameter set 210, the setting value of screen layout of the image parameter is vertical when the depth is shallow and the setting value is horizontal when the depth is deep.

In the image parameter set 210, the trapezoidal scanning is valid when the depth is deep. When the trapezoidal scanning is valid, image quality is lowered since the sound ray interval is broadened in the deep part. In order to complement this, the sound ray density is made high in the deep part. However, when the sound ray density is made high, the frame rate is lowered and followability is lowered, and thus, the time average is made weak in the deep part. By making the depth deep and the time average weak, SNR (signal noise ratio) is lowered, and thus, the dynamic range is lowered. In accordance with this, the gain (offset TGC and offset gain) is changed. In such a way, in the image parameter set 210, image parameters are set so that good ultrasound image data is obtained by mutual cooperation of setting information of a plurality of image parameters.

The image parameters of B-mode of the image parameter set 210 are not limited to these, and may be other image parameters such as transmission waveform, transmission opening, transmission focus, reception frequency, image processing and signal processing. The transmission waveform is a drive signal waveform, for example. The transmission opening is the number of channels of oscillator of the transmission opening of the ultrasound probe 1b. The transmission focus is the focusing distance of the ultrasound probe 1b. The reception frequency is the frequency [MHz] of the reception ultrasound waves (reflected ultrasound waves, echoes). The image processing is information on image processing of edge enhancement and strengthening or weakening the smoothing, for example. The signal processing is information on signal processing such as characteristic coefficient of the dynamic filter, for example.

The image parameter sets 220 . . . have setting values of a plurality of image parameters corresponding to a plurality of depths for a plurality of image modes such as the B-mode and the M-mode similarly to the image parameter set 210.

Especially, the “sound ray density”, the “time average” and the like are used as image parameters not only in the B-mode but also in the color Doppler mode, power Doppler mode and elastography mode. Thus, in the color Doppler mode, power Doppler mode and elastography mode, the setting information such as “sound ray density” and “time average” is set, and stored in the image parameter set of the image mode. The image parameters such as “sound ray density” and “time average” have the action and effect similar to those of the B-mode also in the color Doppler mode, power Doppler mode and elastography mode.

As an image parameter of the image mode other than the B-mode, the “repeat transmission number” is used in the color Doppler mode. Thus, in the color Doppler mode, the setting information of “repeat transmission number” is set and stored in the image parameter set of the color Doppler mode. Specifically, the sensitivity is improved by increasing the setting value of “repeat transmission number” when the depth is shallow, and the frame rate is secured by decreasing the setting value of “repeat transmission number” when the depth is deep.

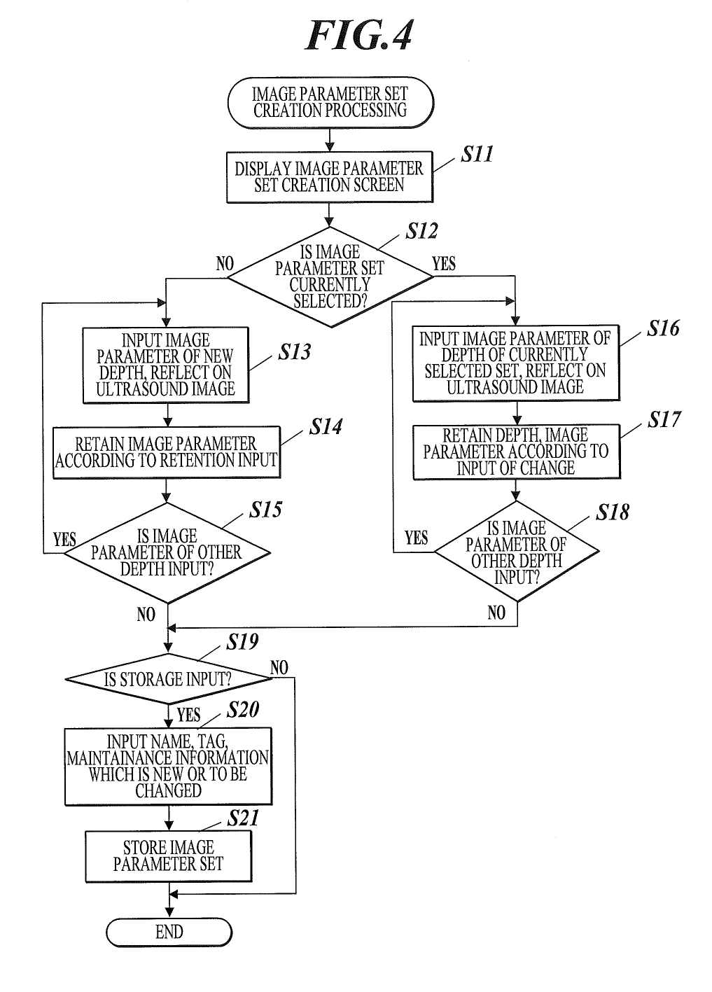

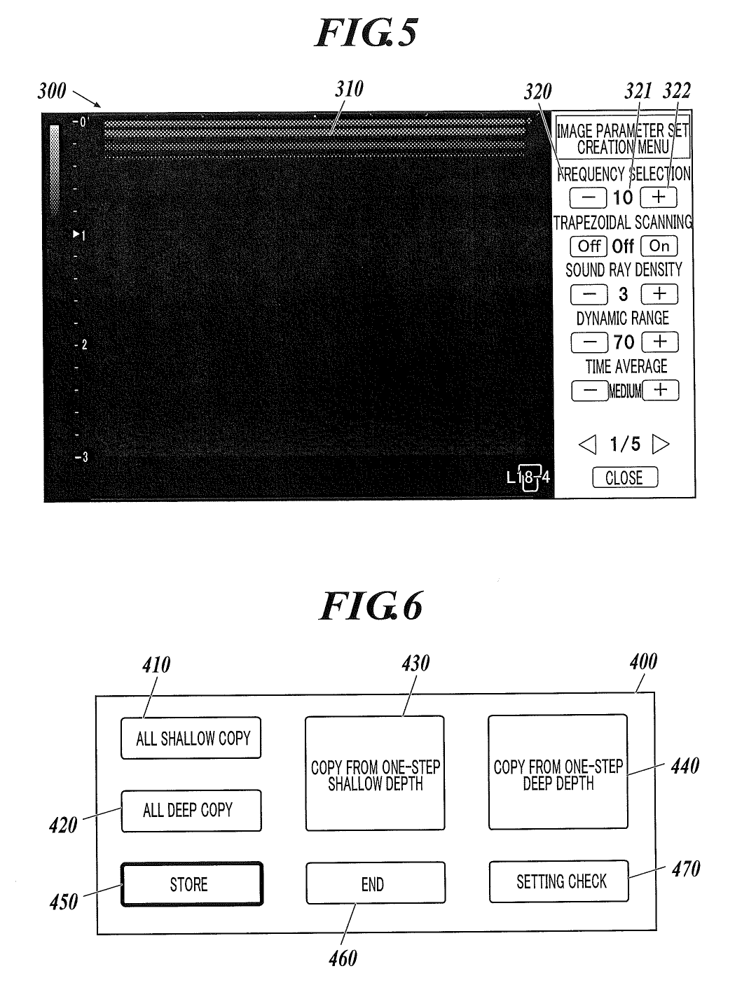

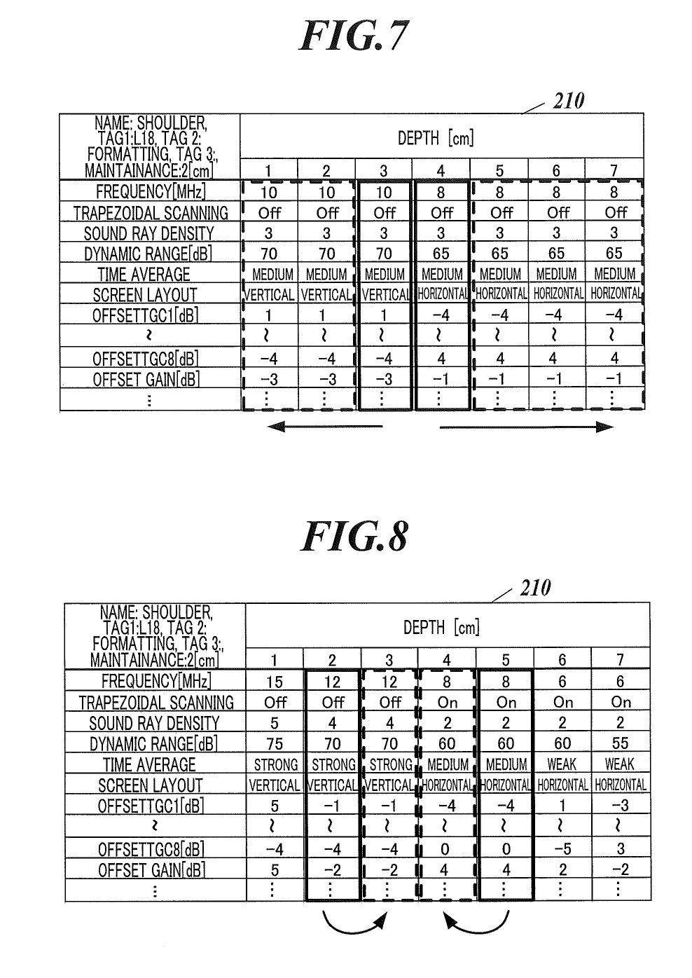

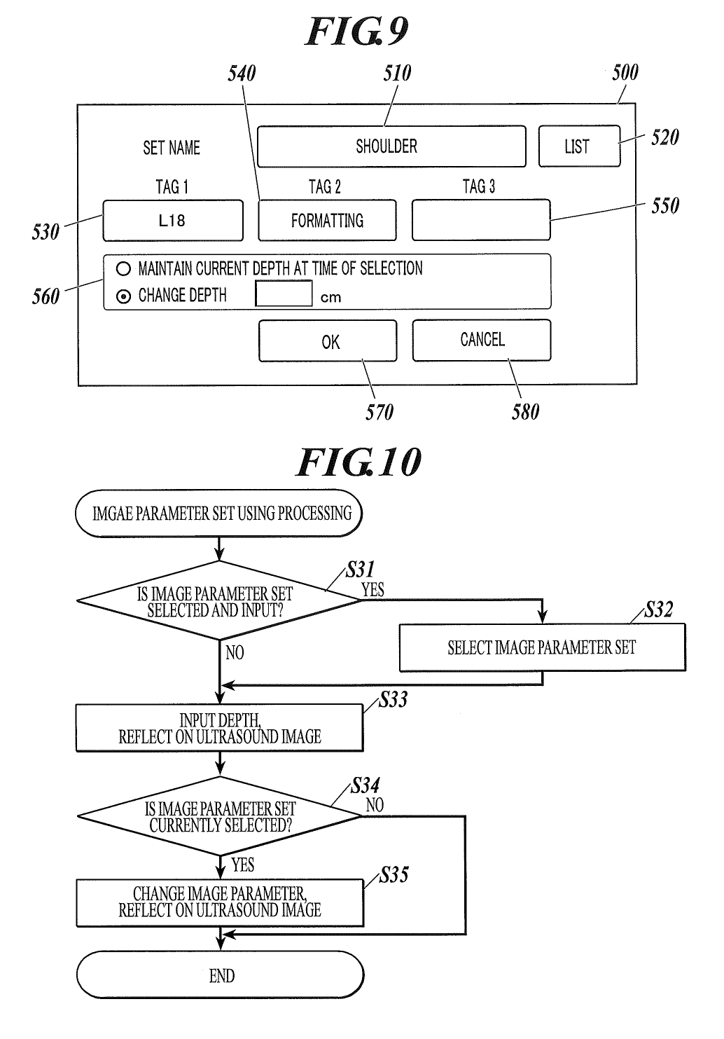

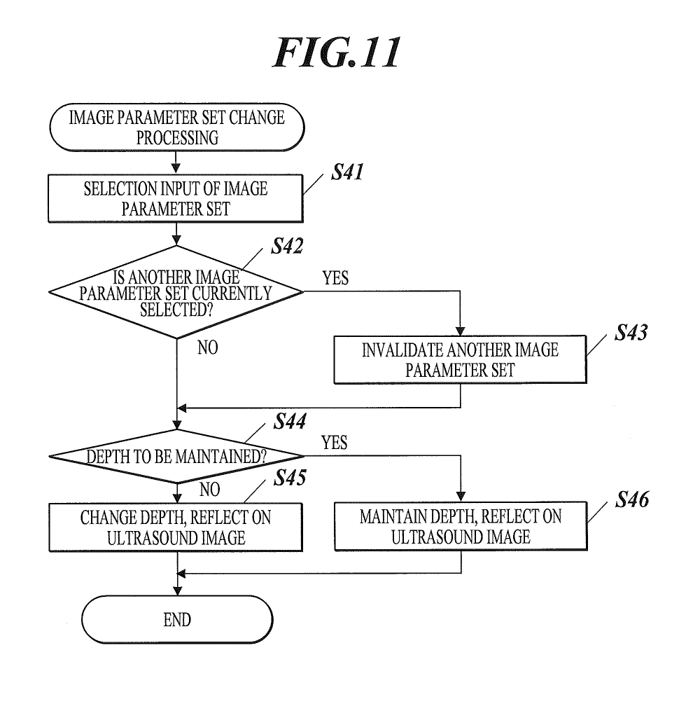

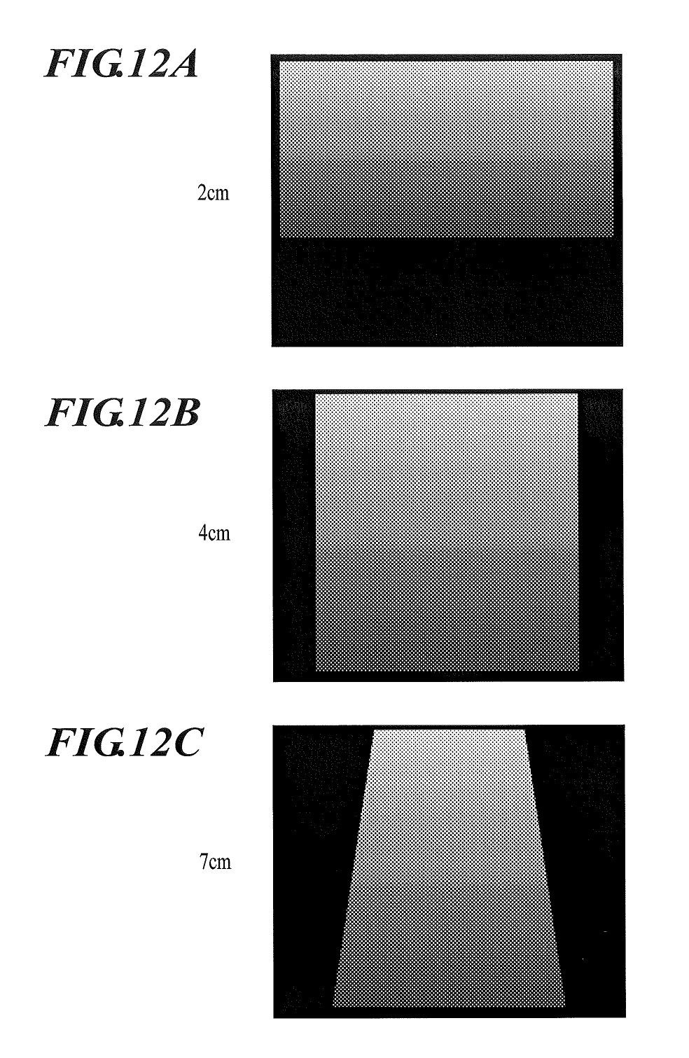

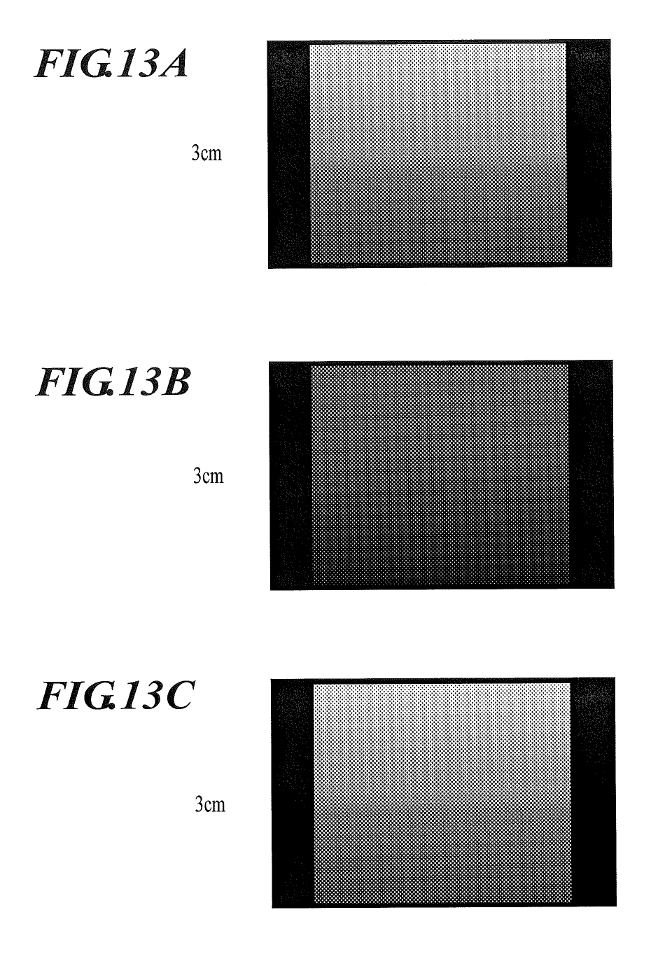

Next, with reference to FIG. 4 to FIG. 13C, the operation of the ultrasound diagnostic device 1 will be described. FIG. 4 is a flowchart showing image parameter set creation processing. FIG. 5 is a view showing an image parameter set creation screen 300. FIG. 6 is a view showing an image parameter copy window 400. FIG. 7 is a view showing an image parameter set 210 of all shallow copy and all deep copy. FIG. 8 is a view showing an image parameter set 210 of one-step shallow copy and one-step deep copy. FIG. 9 is a view showing a storage dialog 500. FIG. 10 is a flowchart showing image parameter set using processing. FIG. 11 is a flowchart showing image parameter set change processing. FIG. 12A is a view showing a B-mode image of the depth 2 [cm] of the image parameter set 210. FIG. 12B is a view showing a B-mode image of the depth 4 [cm] of the image parameter set 210. FIG. 12C is a view showing a B-mode image of the depth 7 [cm] of the image parameter set 210. FIG. 13A is a view showing a B-mode image of the depth 3 [cm] of the image parameter set 210. FIG. 13B is a view showing a B-mode image of the depth 3 [cm] for which the observation site of the image parameter set 210 is changed. FIG. 13C is a view showing a B-mode image of the depth 3 [cm] of the image parameter set 220.

First, with reference to FIG. 4, the image parameter set creation processing executed in the ultrasound diagnostic device 1 will be described. The image parameter set creation processing is processing of newly creating or changing the image parameter set and storing the image parameter set while the operating person such as a doctor is checking the display contents of the image parameter set in the live ultrasound image (live image). A technician or the like as a subject lies on a bed in advance in the examination room provided with the ultrasound diagnostic device 1.

In the ultrasound diagnostic device 1, according to the input of live image display instruction to display the live ultrasound image (live image) in an arbitrary image mode (for example, B-mode) from the operating person via the operation input section 101, the controller 108 causes the transmitter 102 to output the drive signal for live image display to the ultrasound probe 1b put on the subject to emit the transmission ultrasound waves, causes the receiver 103 to receive the reception signal corresponding to the reflected ultrasound waves (echoes) from the ultrasound probe 1b to cause the image generator 104 to generate ultrasound image data, causes the image processor 105 to perform image processing, and causes the display 107 to display the ultrasound image of the live image of the subject via the DSC 106. At this time, in the ultrasound diagnostic device 1, by executing after-mentioned image parameter set using processing or image parameter set change processing, the operating person may select and input the image parameter set of a desired image mode among the image parameter set group 200 so as to display the live image corresponding to the selected image parameter set.

In the ultrasound diagnostic device 1, for example, in response to the trigger that the operating person inputs the desired image mode (for example, B-mode) and execution instruction of the image parameter set creation processing via the operation input section 101, the controller 108 executes the image parameter set creation processing in accordance with the image parameter set creation program stored in the ROM.

As shown in FIG. 4, first, the controller 108 causes the display 107 to display the image parameter set creation screen of the desired image mode which is instructed and input (step S11). In step S11, for example, the image parameter set creation screen 300 of B-mode shown in FIG. 5 is displayed. The image parameter set creation screen 300 has a live image display area 310 and an image parameter input area 320. The live image display area 310 is a display area of the live image. The image parameter input area 320 is the input area of the setting values of a plurality of image parameters. The image parameter input area 320 has, for example, an image parameter value display area 321 to display the setting value and a value input button 322 to receive the input of setting value for each of the plurality of image parameters.

The controller 108 determines whether the image parameter set is currently selected by the operating person's input before the image parameter set creation processing (step S12). If the image parameter set is not currently selected (step S12: NO), as the new image parameter set creation, the controller 108 receives input of a new depth and setting values of a plurality of image parameters in the desired image mode corresponding to the depth via the operation input section 101 and reflects input information (depth, setting values of image parameters in the image mode) on the live image which is currently displayed (step S13). For example, on the image parameter set creation screen 300, the depth is changed by dial input of the operation input section 101, and each time the value input button 322 is touched and input, the controller 108 appropriately controls the transmitter 102, the receiver 103, the image generator 104, the image processor 105 and the like according to the input depth and image parameters, to display the live image corresponding to the input depth and the image parameters in the live image display area 310.

The controller 108 retains the depth and the plurality of image parameters input in step S13 according to the retention input from the operating person via the operation input section 101 (step S14). The controller 108 determines whether the operating person inputs an image parameter of any other depth via the operation input section 101 (step S15). If there is input of an image parameter of any other depth (step S15: YES), the processing proceeds to step S13.

If the image parameter set is currently selected (step S12: YES), as image parameter set change, the controller 108 reads out the currently-selected image parameter set of the desired image mode from the storage 109 via the operation input section 101, receives input of the depth to be changed in the image parameter set which is currently selected and setting values of a plurality of image parameters in the desired image mode corresponding to the depth, and reflects the input information (depth, setting values of image parameters in the image mode) on the live image which is currently displayed (step S16). For example, on the image parameter set creation screen 300, the depth is changed by dial input of the operation input section 101, and the setting value of each of the image parameters in the image parameter set which is currently selected is displayed in the image parameter value display area 321, and each time the value input button 322 is touched and input, the controller 108 displays the live image corresponding to the input depth and the image parameter in the live image display area 310 similarly to step S13.

The controller 108 retains the depth input in step S13 and the plurality of image parameters according to the retention input from the operating person via the operation input section 101 (step S17). The controller 108 determines whether there is input of an image parameter of any other depth in the image parameter set which is currently selected from the operating person via the operation input section 101 (step S18). If there is input of image parameter of other depth (step S18: YES), the processing proceeds to step S16.

Though the setting values of the plurality of image parameters are input for each depth in steps S16 to S18, there is a case where a same setting value is set to a plurality of image parameters at different depths. Thus, in step S16, the image parameter copy window 400 shown in FIG. 6 may be displayed on the display 107 so that a same setting value is copied to the plurality of image parameters at different depths.

The image parameter copy window 400 has an all shallow copy button 410, an all deep copy button 420, a one-step shallow copy button 430, a one-step deep copy button 440, a storage button 450, an end button 460 and a setting check button 470.

The all shallow copy button 410 is a button for receiving execution input to copy the setting values of the plurality of image parameters at the currently-selected depth to the setting values of the plurality of image parameters at all the depths which are shallower than the currently-selected depth. For example, in the image parameter set 210 in FIG. 3, when the all shallow copy button 410 is touch-input while the depth 3 [cm] is selected, as shown in FIG. 7, the setting values of the plurality of image parameters at the depth 3 [cm] are retained so as to be copied to the setting values of the plurality of image parameters at the depths 1 and 2 [cm] which are shallower than 3 [cm].

The all deep copy button 420 is a button for receiving execution input to copy the setting values of the plurality of image parameters at the currently-selected depth to the setting values of the plurality of image parameters at all the depths which are deeper than the currently-selected depth. For example, in the image parameter set 210 in FIG. 3, when the all shallow copy button 410 is touch-input while the depth 4 [cm] is selected, as shown in FIG. 7, the setting values of the plurality of image parameters at the depth 4 [cm] are retained so as to be copied to the setting values of the plurality of image parameters at the depths 5, 6 and 7 [cm] which are deeper than 4 [cm].

The one-step shallow copy button 430 is a button for receiving execution input to copy the setting values of the plurality of image parameters at the depth which is one-step shallower than the currently-selected depth to the setting values of the plurality of image parameters at the currently-selected depth. For example, in the image parameter set 210 in FIG. 3, when the one-step shallow copy button 430 is touch-input while the depth 3 [cm] is selected, as shown in FIG. 8, the setting values of the plurality of image parameters at the depth 2 [cm] which is one-step shallower than the depth 3 [cm] are retained so as to be copied to the setting values of the plurality of image parameters at the depth 3 [cm].

The one-step deep copy button 440 is a button for receiving execution input to copy the setting values of the plurality of image parameters at the depth which is one-step deeper than the currently-selected depth to the setting values of the plurality of image parameters at the currently-selected depth. For example, in the image parameter set 210 in FIG. 3, when the one-step deep copy button 440 is touch-input while the depth 4 [cm] is selected, as shown in FIG. 8, the setting values of the plurality of image parameters at the depth 5 [cm] which is one-step deeper than the depth 4 [cm] are retained so as to be copied to the setting values of the plurality of image parameters at the depth 4 [cm].

The storage button 450 is a button for receiving execution input of storage of the input image parameter set. The end button 460 is a button for receiving execution input of end of the image parameter set creation processing. The setting check button 470 is a button for receiving execution input of display of the image parameter retained in step S17 to the display 107 for check.

In step S16, the controller 108 may cause the display 107 to display the table of the image parameter set which is currently selected so as to receive change input of the setting values of the image parameters in the table of the image parameter set which is displayed. Similarly, in step S13, the operating person may input setting values of the image parameters via the operation input section 101 in the table of the image parameter set displayed on the display 107.

Returning to FIG. 4, if there is no input of image parameter at any other depth (step S15: NO) or (step S18: NO), the controller 108 determines whether the operating person inputs execution of storage via the operation input section 101 (step S19). In step S19 after step S18, for example, the above determination is made by whether the storage button 450 is touch-input or not. If the execution of storage is not input (step S19: NO), the image parameter set creation processing ends.

If execution of storage is input (step S19: YES), the controller 108 receives input of the name, tags and maintenance information of the image parameter set to be newly created or changed from the operating person via the operation input section 101 (step S20). In step S20, for example, the storage dialog 500 shown in FIG. 9 is displayed, and the input is performed via the storage dialog 500. The storage dialog 500 has a name input field 510, tag input fields 530, 540 and 550, a maintenance information input field 560, an OK button 570 and a cancel button 580.

The name input field 510 is an input field of the name of image parameter set to be newly created or changed. The tag input fields 530, 540 and 550 are input field of tags 1, 2 and 3 of the image parameter set to be newly created or changed. The maintenance information input field 560 is an input field including radio buttons for receiving input of whether to maintain the last depth and the depth after change in a case of not maintaining (that is, changing) the depth at the time of newly selecting the image parameter set to be newly created or changed. In a case of proceeding through step S16, the name, tags 1, 2 and 3 and maintenance information of the currently-selected image parameter set read out in step S16 are displayed as default so as to be able to be changed and input in the name input field 510, the tag input fields 530, 540 and 550 and maintenance input field 560.

The OK button 570 is a button for receiving execution input of storage and overwriting of input information in the name input field 510, the tag input fields 530, 540 and 550 and the maintenance information input field 560. The cancel button 580 is a button for receiving execution input of cancellation of input (storage) in the name input field 510, the tag input fields 530, 540 and 550 and the maintenance information input field 560.

The controller 108 stores the image parameter set which includes a plurality of image parameters at a plurality of depths retained in step S14 or S17 in the storage 109 so as to be associated with the name, tags 1, 2 and 3 and the maintenance information input in step S20 (step S21), and ends the image parameter set creation processing. In step S21, the stored image parameter set may be displayed on the display 107.

In steps S16 to 18 in the image parameter set creation processing, the operating person may specify (specify by input via the operation input section 101) the image parameter to be changed or to be maintained (not changed) in the image parameter set which is currently selected, and input and change only the setting information of the image parameter to be changed.

Next, with reference to FIG. 10, the image parameter set using processing executed in the ultrasound diagnostic device 1 will be described. The image parameter set using processing is processing of selecting the image parameter set from the image parameter set group 200 and reflecting the selected image parameter set in the live image. A patient as the subject lies on a bed in advance in the examination room provided with the ultrasound diagnostic device 1.

Similarly to the image parameter set creation processing, in the ultrasound diagnostic device 1, the live image of a desired image mode (for example, B-mode) which is input is generated and displayed on the display 107 according to the input from the operating person via the operation input section 101.

In the ultrasound diagnostic device 1, for example, in response to a trigger that the operating person inputs the desired image mode and execution instruction of image parameter set using processing via the operation input section 101, the controller 108 executes the image parameter set using processing in accordance with the image parameter set using program stored in the ROM.

As shown in FIG. 10, first, the controller 108 receives the selection input of the image parameter set of the image mode which is instructed and input from the operating person via the operation input section 101, and determines whether the image parameter set is selected and input (step S31). In step S31, for example, the controller 108 displays the search screen of the image parameter set on the display 107, receives input of the image mode, the keyword of name and tags of the image parameter set from the operating person via the operation input section 101, searches in the image parameter set group 200 of the storage 109 with the input image mode, keyword and tags, displays the image parameter sets as the search results, and receives the selection input of one image parameter set among the search results from the operating person. In step S31, the operation input section 101 may receive selection of one image parameter set via physical keys or buttons (displayed buttons) of the touch panel to which a plurality of image parameter sets are assigned.

If the image parameter set is selected and input (step S31: YES), the controller 108 selects (sets) the image parameter set which was selected and input in step S31 (step S32). After step S32, or if the image parameter set is not selected and input (step S31: NO), the controller 108 receives input of the depth from the operating person via the operation input section 101, according to the input depth, appropriately controls the transmitter 102, the receiver 103, the image generator 104, the image processor 105 and the like to display the live image on the display 107 corresponding to the input depth of the image mode which was instructed and input (step S33).

The controller 108 determines whether the image parameter set is currently selected (valid) in step S32 (step S34). If the image parameter set is not currently selected (step S34: NO), the image parameter set using processing ends. If the image parameter set is currently selected (step S34: YES), according to the depth input in step S33 and the image parameter set selected in step S32, the controller 108 appropriately controls the transmitter 102, the receiver 103, the image generator 104, the image processor 105 and the like to cause the display 107 to display the live image corresponding to the input depth and the image parameters of the image mode which was instructed and input (step S35), and ends the image parameter set using processing.

Next, with reference to FIG. 11, the image parameter set change processing executed in the ultrasound diagnostic device 1 will be described. The image parameter set change processing is processing of changing the image parameter set selected from the image parameter set group 200 and reflecting it on the live image. The image parameter set using processing (or image parameter set change processing) of the desired image mode is executed in advance.

In the ultrasound diagnostic device 1, for example, in response to a trigger that the operating person inputs the execution instruction of image parameter set change processing of the desired image mode via the operation input section 101, the controller 108 executes the image parameter set change processing in accordance with the image parameter set changing program stored in the ROM.

As shown in FIG. 11, first, similarly to step S31 in FIG. 10, the controller 108 receives the selection input of the image parameter set of the image mode which is instructed and input from the operating person via the operation input section 101, and selects the image parameter set which is selected and input (step S41). The controller 108 determines whether another image parameter set has been selected immediate before step S41 (step S42). If another image parameter set has been selected (step S42: YES), the controller 108 invalidate the another image parameter set which has been selected (step S43).

If another image parameter set has not been selected (step S42: NO) or after execution of step S43, the controller 108 refers to maintenance information of the image parameter set selected in step S41, and determines whether to maintain the depth (step S44). If the depth is not maintained (step S44: NO), according to the depth of maintenance information referred to in step S44 and the image parameter set which is currently selected, the controller 108 appropriately controls the transmitter 102, the receiver 103, the image generator 104, the image processor 105 and the like to cause the display 107 to display the live image corresponding to the depth and the image parameter of the image mode which was instructed and input (step S45), and ends the image parameter set change processing.

If the depth is maintained (step S44: YES), according to the depth which was selected immediately before in the another image parameter set and the image parameter set which is currently selected, the controller 108 appropriately controls the transmitter 102, the receiver 103, the image generator 104, the image processor 105 and the like to cause the display 107 to display the live image corresponding to the depth and the image parameter which is currently selected of the image mode which was instructed and input (step S46), and ends the image parameter set change processing.

Here, the description will be made for an example in which the image parameter set using processing is repeatedly executed so that the live image of the B-mode image is displayed, the image parameter set 210 is selected, and the depth is changed while using the image parameter set 210. In the first image parameter set using processing, when the image parameter set 210 is selected in step S32 and the depth 2 [cm] is input in step S33, for example, in step S35, a rectangular B-mode image at the depth 2 [cm] shown in FIG. 12A is displayed.

In the second image parameter set using processing, when the selection input is not performed in step S32 (same image parameter set 210 is used) and the depth 4 [cm] is input in step S33, for example, a rectangular B-mode image at the depth 4 [cm] shown in FIG. 12B is displayed in step S35.

In the third image parameter set using processing, when the selection input is not performed in step S32 (same image parameter set 210 is used) and the depth 7 [cm] is input in step S33, for example, a trapezoidal B-mode image at the depth 7 [cm] shown in FIG. 12C is displayed in step S35.

Here, the description will be made for an example in which the image parameter set using processing and the image parameter set change processing are executed so that the live image of the B-mode image is displayed, the image parameter set 210 is changed to the image parameter set 220, and the depth is maintained. In the image parameter set using processing, when the image parameter set 210 is selected in step S32 and the depth 3 [cm] is input in step S33, for example, in step S35, a rectangular B-mode image at the depth 3 [cm] shown in FIG. 13A is displayed.

In a case where the ultrasound probe 1b is moved in order to change the observation site, for example, the rectangular B-mode image at the depth 3 [cm] shown in FIG. 13B is displayed. However, the B-mode image in FIG. 13B is highly attenuation image, and thus, it cannot be observed. Thus, the image parameter set change processing is executed, the image parameter set 220 is input in step S41, and when the depth 3 [cm] is maintained in step S44, for example, in step S46, the rectangular B-mode image at the depth 3 [cm] shown in FIG. 13C is displayed, leading to the B-mode image with attenuation corresponding to the observation site. Here, the image parameter set 220 corresponds to the observation site after change and the maintenance information is On.

As described above, according to the embodiment, the ultrasound diagnostic device 1 includes: a transmitter 102 which outputs a drive signal to an ultrasound probe 1b transmitting and receiving ultrasound waves; a receiver 103 which obtains a reception signal from the ultrasound probe 1b; an image generator 104 which generates ultrasound image data from the reception signal; an image processor 105 which performs image processing to the generated ultrasound image data; an operation input section 101 which receives input of a depth of the ultrasound image to be displayed; and a controller 108 which obtains a plurality of image parameters which is set in advance corresponding to the input depth, and, according to the obtained plurality of image parameters, controls the transmitter 102, the receiver 103, the image generator 104 and the image processor 105 to generate the ultrasound image data corresponding to the input depth.

Thus, it is possible to change a plurality of image parameters according to the change of depth, and easily obtain good ultrasound image data corresponding to the plurality of image parameters without troublesome operation.

The plurality of image parameters is image parameters corresponding to at least one image mode among a B-mode, an M-mode, a color Doppler mode, a power Doppler mode, a CW mode and an Elastography mode. Thus, it is possible to change the plurality of image parameters according to the change in depth for various image modes.

The controller 108 obtains a plurality of image parameters corresponding to the input depth from the image parameter sets 210, 220 . . . which are tables having a plurality of preset image parameters corresponding to a plurality of depths stored in the storage 109. Thus, by using the image parameter sets, it is possible to easily control image parameters and improve visibility in checking the setting values of the respective image parameters for each depth.

The storage 109 stores a plurality of types of image parameter sets 210, 220 . . . (image parameter set group 200). The operation input section 101 receives input of the depth of the ultrasound image data to be generated and selection input of one image parameter set from the plurality of types of image parameter sets. The controller 108 obtains the plurality of image parameters corresponding to the input depth from the image parameter set which was selected and input. Thus, it is possible to apply the optimum image parameters corresponding to the condition such as the type of the ultrasound probe 1b, the site and the subject to the ultrasound image data.

The operation input section 101 receives selection of one image parameter set via physical keys or buttons (displayed buttons) of a touch panel to which a plurality of image parameter sets are assigned. Thus, it is possible to simply select and switch the image parameter set.

When a selection input of changing the image parameter set is performed by the operation input section 101, the controller 108 maintains the depth before selection input according to the maintenance information, and obtains a plurality of image parameters corresponding to the depth before the selection input in the image parameter set which was selected and input. Thus, only the image parameters are changed, and it is not necessary to perform setting again due to the depth becoming an unintended value.

The operation input section 101 receives input of setting values of a plurality of image parameters for each depth of the image parameter set. The controller 108 stores the image parameter set corresponding to the setting values of the plurality of image parameters for each input depth in the storage 109. Thus, the operating person can freely customize the image parameter set and obtain ultrasound image data corresponding to the operating person's preference.

According to the setting values of a plurality of image parameters of the input depth, the controller 108 controls the transmitter 102, the receiver 103, the image generator 104 and the image processor 105 to generate the ultrasound image data corresponding to the input depth and cause the display 107 to display the generated data. Thus, it is possible to freely customize the image parameter set while visually checking whether a desired ultrasound image is obtained.

One of the plurality of image parameters is trapezoidal scanning, and the setting value is set in the image parameter set so that the trapezoidal scanning is invalid or the angle is small when the depth is shallow, and the trapezoidal scanning is valid or the angle is large when the depth is deep. When the depth is shallow, the area is located out of the display area even if the trapezoidal scanning is performed, and the effect of view expansion cannot be obtained. Thus, the image quality can be improved by invalidating the trapezoidal scanning or reducing the angle and increasing the density between the scanning lines. On the other hand, when the depth is deep, it is possible to improve the effect of view expansion by validating the trapezoidal scanning or increasing the angle and having the expanded view area by the trapezoidal scanning within the display area.

One of the plurality of image parameters is a sound ray density, and the setting value is set to the image parameter set so that the sound ray density is high when the depth is shallow and the sound ray density is low when the depth is deep. When the sound ray density becomes higher, the frame rate is lowered though the image quality is high. When the sound ray density becomes lower, the frame rate is improved though the image quality is lowered. The frame rate is improved when the depth is shallow, and the frame rate is lowered when the depth is deep. This leads to a good solution to these trade-off relationships.

One of the plurality of image parameters is a time average, and the setting value is set to the image parameter set so that the time average is strong when the depth is shallow and the time average is weak when the depth is deep. When the depth is shallow, the frame rate is improved and followability is improved. When the depth is deep, the frame rate is lowered and followability is lowered. The followability is lowered when the time average becomes stronger, and the followability is improved when the time average becomes weaker. This leads to a good solution to these trade-off relationships.

One of the plurality of image parameters is a dynamic range, and the setting value is set to the image parameter set so that the dynamic range is high when the depth is shallow and the dynamic range is low when the depth is deep. The SNR is high at the shallow part of the ultrasound image, and the SNR is low at the deep part of the ultrasound image. Thus, it is possible to make the dynamic range higher in order to display from a strong signal to a weak signal when the SNR is high, and it is possible to make the dynamic range lower in order to display a weak signal with a high brightness when the SNR is low.

One of the plurality of image parameters is a screen layout setting in double-screen display, and the setting value is set to the image parameter set so that the screen layout setting is vertical when the depth is shallow and the screen layout is horizontal when the depth is deep. The ultrasound image is horizontally long when the depth is shallow, and the ultrasound image is vertically long when the depth is deep. Thus, it is possible to use vertical screen layout in the direction of the horizontally long ultrasound image when the depth is shallow, and use horizontal screen layout in the direction of the vertically long ultrasound image when the depth is deep.

One of the plurality of image parameters is offset TGC. The controller 108 causes the image processor 105 to provide the addition value of the gain of offset TGC of input depth and the gain of normal TGC to the ultrasound image data corresponding to the input depth. Thus, it is possible to correct ununiformity in image brightness changing in the depth direction by the offset TGC while maintaining conventional use of TGC of normal operation.

One of the plurality of image parameters is offset gain. The controller 108 causes the image processor 105 to provide the addition value of the offset gain of input depth and the gain by normal operation to the ultrasound image data corresponding to the input depth. Thus, it is possible to suppress the depth change and the brightness change by the image parameter set while maintaining conventional use of gain at the time of normal operation.

The description in the above embodiment is an example of an ultrasound diagnostic device, an image parameter setting method and a storage medium which are preferred according to the present invention, and the present invention is not limited to this example.

For example, though the image parameter set group 200 is stored in the storage 109 of the ultrasound diagnostic device 1 in the embodiment, the present invention is not limited to this. For example, the ultrasound diagnostic device 1 may include a reading/writing section of an external media such as a USB (Universal Serial Bus) memory and an SD (Secure Digital) card so that the image parameter set group 200 can be stored in the external media. The external media storing the image parameter set group 200 is connected to another ultrasound diagnostic device, and the image parameter set group 200 is copied to the storage of the another ultrasound diagnostic device to be used.

When the image parameter set to be used in the image parameter set group 200 is not selected, the plurality of image parameters may reflect preset setting values in the ultrasound image. The preset values are information group having setting values of a plurality of image parameters (including the depth in this case), body mark information and text information for each image mode such as the B-mode and a shared image mode.

At the same time as predetermined preset selection, it may be set whether to validate a predetermine image parameter set.

In the configuration of displaying a table of the image parameter set, a difference between setting values may be emphasized by coloring or the like when there is a difference which is a predetermined threshold or more between a setting value of an image parameter at one depth and a setting value of an image parameter at a depth next to the one depth (for example, the depth which is one-step shallower or deeper than the one depth).

Though the image parameter set is in a form of table in the embodiment, the present invention is not limited to this. The image parameter set may be anything as long as the setting value of the image parameter can be changed for each depth, and the image parameter set may be a function of the image parameter to which the depth is input. Furthermore, there may be a plurality of functions for a same image parameter.

The image parameter set includes setting values of a plurality of image parameters of a plurality of image modes. When a part of one image parameter set is copied to create another image parameter set, setting values of a plurality of image parameters of image modes which are a part of the one image parameter set may be copied to the another image parameter set.

Though the description has been made for creation, use and change of the image parameter set which is other than the depth and corresponding to the depth, the above contents of the embodiment may be used for a parameter other than the depth, the parameter serving as an opportunity to change the image parameter set.

As for the detailed configurations and detailed operations of the components forming the ultrasound diagnostic device 1 in the embodiment, modifications can be appropriately made within the scope of the present invention.

Although embodiments of the present invention have been described and illustrated in detail, the disclosed embodiments are made for purposes of illustration and example only and not limitation. The scope of the present invention should be interpreted by terms of the appended claims.

The entire disclosure of Japanese patent Application No. 2017-147649, filed on 31 Jul. 2017, and No. 2018-005313, filed on 17 Jan. 2018, are incorporated herein by reference in its entirety.

What is claimed is:

1. An ultrasound diagnostic device, comprising:
a transmitter which outputs a drive signal to an ultrasound probe that transmits and receives ultrasound waves;
a receiver which obtains a reception signal from the ultrasound probe;
a first hardware processor which generates ultrasound image data from the reception signal and performs image processing to the generated ultrasound image data;
an operator which receives input of a depth of an ultrasound image to be displayed; and
a second hardware processor which obtains a plurality of preset image parameters corresponding to the input depth, and controls the transmitter, the receiver and the first hardware processor to generate the ultrasound image data corresponding to the input depth according to the obtained plurality of image parameters.

2. The ultrasound diagnostic device according to claim 1, wherein the plurality of image parameters are image parameters corresponding to at least one image mode among a B-mode, an M-mode, a color Doppler mode, a power Doppler mode, a CW mode and an Elastography mode.

3. The ultrasound diagnostic device according to claim 1, wherein the second hardware processor obtains the plurality of image parameters corresponding to the input depth from an image parameter set which is stored in a storage and has a plurality of preset image parameters corresponding to a plurality of depths.

4. The ultrasound diagnostic device according to claim 3, wherein
the storage stores a plurality of types of image parameter sets,
the operator receives input of a depth of ultrasound image data to be generated and selection input of an image parameter set from among the plurality of types of image parameter sets, and
the second hardware processor obtains the plurality of image parameters corresponding to the input depth from the image parameter set which is selected and input.

5. The ultrasound diagnostic device according to claim 3, wherein the operator receives selection of an image parameter set via a key or a button of a touch panel to which a plurality of image parameter sets are assigned.

6. The ultrasound diagnostic device according to claim 3, wherein, when the operator receives selection input of changing the image parameter set, the second hardware processor maintains the depth before the selection input, and obtains a plurality of image parameters corresponding to the depth before the selection input in the image parameter set which is selected and input.

7. The ultrasound diagnostic device according to claim 3, wherein,
the operator receives input of setting values of a plurality of image parameters for each depth in the image parameter set, and
the second hardware processor stores the image parameter set corresponding to the input setting values of the plurality of image parameters for each depth in the storage.

8. The ultrasound diagnostic device according to claim 7, wherein, according to the plurality of input image parameters for each depth, the second hardware processor controls the transmitter, the receiver and the first hardware processor to generate the ultrasound image data corresponding to the input depth and display the generated ultrasound image data on a display.

9. The ultrasound diagnostic device according to claim 1, wherein one of the plurality of image parameters is trapezoidal scanning, and a setting value is set such that the trapezoidal scanning is invalid or an angle is small when the depth is shallow and the trapezoidal scanning is valid or the angle is large when the depth is deep.

10. The ultrasound diagnostic device according to claim 1, wherein one of the plurality of image parameters is a sound ray density, and a setting value is set such that the sound ray density is high when the depth is shallow and the sound ray density is low when the depth is deep.

11. The ultrasound diagnostic device according to claim 1, wherein one of the plurality of image parameters is a time average, and a setting value is set such that the time average is strong when the depth is shallow and the time average is weak when the depth is deep.

12. The ultrasound diagnostic device according to claim 1, wherein one of the plurality of image parameters is a dynamic range, and a setting value is set such that the dynamic range is high when the depth is shallow and the dynamic range is low when the depth is deep.

13. The ultrasound diagnostic device according to claim 1, wherein one of the plurality of image parameters is a screen layout setting, and a setting value is set such that the screen layout setting is vertical when the depth is shallow and the screen layout setting is horizontal when the depth is deep.

14. The ultrasound diagnostic device according to claim 1, wherein one of the plurality of image parameters is offset TGC, and the second hardware processor controls the first hardware processor to provide a value obtained by adding a gain of offset TGC of the input depth and a gain of normal TGC to the ultrasound image data corresponding to the input depth.

15. The ultrasound diagnostic device according to claim 1, wherein one of the plurality of image parameters is an offset gain, and the second hardware processor controls the first hardware processor to provide a value obtained by adding an offset gain of the input depth and a gain by a normal operation to the ultrasound image data corresponding to the input depth.

16. An image parameter setting method in an ultrasound diagnostic device including: a transmitter which outputs a drive signal to an ultrasound probe that transmits and receives ultrasound waves; a receiver which obtains a reception signal from the ultrasound probe; and a hardware processor which generates ultrasound image data from the reception signal and performs image processing to the generated ultrasound image data, the method comprising:
a step of receiving input of a depth of an ultrasound image to be displayed; and
a step of obtaining a plurality of preset image parameters corresponding to the input depth, and controlling the transmitter, the receiver and the hardware processor to generate the ultrasound image data corresponding to the input depth according to the obtained plurality of image parameters.

17. A computer readable storage medium storing a program for causing a computer to function as:
a transmitter which outputs a drive signal to an ultrasound probe that transmits and receives ultrasound waves;
a receiver which obtains a reception signal from the ultrasound probe;
a first hardware processor which generates ultrasound image data from the reception signal and performs image processing to the generated ultrasound image data;
an operator which receives input of a depth of an ultrasound image to be displayed; and
a second hardware processor which obtains a plurality of preset image parameters corresponding to the input depth, and controls the transmitter, the receiver and the first hardware processor to generate the ultrasound image data corresponding to the input depth according to the obtained plurality of image parameters.
